(19) United States
(12) Patent Application Publication
(10) Pub. No.: US 2002/0034487 A1
(43) Pub. Date: Mar. 21, 2002

(54) Detergent cosmetic compositions comprising a specific amphoteric starch, and uses thereof

(76) Inventors: Mireille Maubru, Chatou (FR); Bernard Beauquey, Clichy (FR); Veronique Douin, Paris (FR)

Correspondence Address: FINNEGAN, HENDERSON, FARABOW,, GARRETT & DUNNER, L.L.P., 1300 I Street, N.W., Washington, DC, 20005-3315, US

(21) Appl. No.: 09/759,530

(22) Filed: Jan. 16, 2001

(30) Foreign Application Priority Data
Jan. 13, 2000 (FR) 00 00411

(51) Int. Cl. A61K 7/075; A61K 7/11

(52) U.S. Cl. 424/70.13; 424/70.21

(57) ABSTRACT

Detergent cosmetic compositions comprising a washing base and at least one amphoteric starch. This combination can give at least one cosmetic property such as, ease of styling, lightness and suppleness of treated hair, and can have good working properties such as good intrinsic washing power and good foaming power. These compositions can be used for washing and/or conditioning a keratin material, such as the hair or the skin.

The present invention relates to novel cosmetic compositions with improved properties, intended both for cleaning and conditioning keratin materials such as the hair, and comprising, in a cosmetically acceptable aqueous vehicle, a washing base and at least one specific amphoteric starch. The present invention also relates to the use of novel cosmetic compositions comprising, in a cosmetically acceptable aqueous vehicle, a washing base and at least one specific amphoteric starch in cosmetic applications.

It is common to use detergent compositions (such as shampoos) based on surfactants such as anionic, nonionic and amphoteric surfactants to clean and wash keratin materials such as the hair. These compositions are applied to wet hair and the lather generated by massaging or rubbing with the hands makes it possible to remove, after rinsing with water, the various types of soiling which may be initially present on the hair.

While these base compositions are of good washing power, the intrinsic cosmetic properties associated with them may be undesirable, owing, for example, to the fact that the relatively aggressive nature of such a cleaning treatment can, in the long run, lead to more or less pronounced damage to the hair fiber, this damage being associated, for example, with the gradual removal of the lipids or proteins contained in or on the surface of this fiber.

Thus, in order to improve at least one cosmetic property of the above detergent compositions, for example, those which are to be applied to sensitized hair (i.e. hair which has been damaged or made brittle, for example under the chemical action of atmospheric agents and hair treatments such as permanent-waving, dyeing and bleaching), it is common to introduce additional cosmetic agents known as conditioners into these compositions, these conditioners being intended mainly to repair or limit the harmful or undesirable effects which may be induced by the various treatments and aggressions to which the hair fibers are subjected more or less repeatedly. These conditioners may, of course, also improve the cosmetic behavior of natural hair.

It has been recommended to use combinations of nonionic starch with anionic surfactants to formulate shampoos. However, these combinations may not lead to satisfactory cosmetic results.

The combination of an amphoteric starch with soap has also been recommended for shaving foam compositions, but these compositions may not lead to sufficiently powerful detergent properties and the use of soap to wash the hair may present major drawbacks such as dulling of the fiber.

Thus, after considerable research conducted in this matter, the inventors have found that by using a washing base and at least one amphoteric starch, it is possible to obtain a composition that has at least one excellent cosmetic property, for example, ease of styling, lightness and suppleness of treated hair, and has at least one good working property, for example, good intrinsic washing power and good foaming power.

The compositions in accordance with the present invention can give the hair, after rinsing, a noteworthy treating effect which is manifested, for example, by the provision of lightness, hold and suppleness.

At least one of these discoveries forms the basis of the present invention.

Thus, one subject of the present invention is a detergent and conditioning cosmetic composition comprising, in a cosmetically acceptable aqueous medium, a washing base and at least one amphoteric starch defined below, wherein the composition is free of fatty acid soaps.

Another subject of the present invention is a detergent cosmetic composition comprising, in a cosmetically acceptable aqueous medium, a washing base and at least one amphoteric starch defined below, wherein the composition is free of fatty acid soaps. A further subject of the present invention is a conditioning cosmetic composition comprising, in a cosmetically acceptable aqueous medium, a washing base and at least one amphoteric starch defined below, wherein the composition is free of fatty acid soaps.

Yet another subject of the present invention is the cosmetic use of a cosmetic composition according to the present invention for cleaning and/or removing make-up from keratin materials such as the hair and the skin. A further subject of the present invention is the cosmetic use of a cosmetic composition according to the present invention for conditioning keratin materials such as the hair and the skin. An additional subject of the present invention is the use of a cosmetic composition according to the present invention as a shampoo for keratin materials.

As used herein, the expression “fatty acid soap” refers to salts of alkali metals, salts of alkaline-earth metals, fatty amines and C₁₀-C₁₈ fatty acids.

As used herein, the expression “free of fatty acid soaps” means that fatty acid soaps are present in an amount ranging for example from 0% to 1% by weight, such as from 0% to 0.1% by weight, relative to the total weight of the final composition.

As used herein, the expression “detergent composition” refers to a composition with washing power that cleans keratin materials such as hair.

As used herein, the expression “conditioning composition” refers to a composition that repairs or limits the harmful or undesirable effects on keratin materials such as hair, which may be induced by various treatments and aggressions to which the keratin materials are subjected.

The compositions in accordance with the present invention comprise a washing base, which is generally aqueous. The washing base comprises at least one surfactant having washing power. The at least one surfactant forming the washing base may be chosen from anionic, amphoteric and nonionic detergent surfactants.

For example, in one embodiment the washing base comprises at least one anionic surfactant. In another embodiment the washing base comprises at least one anionic surfactant and at least one amphoteric surfactant. In yet another embodiment the washing base comprises at least one anionic surfactant and at least one nonionic surfactant.

In one embodiment according to the present invention, the washing base can be present in an amount ranging for example from 4% to 50% by weight, such as from 6% to 35% by weight, and further such as from 8% to 25% by weight, relative to the total weight of the final composition.

The surfactants which are suitable for carrying out the present invention can, for example, include the following:

(i) Anionic surfactant(s):

Representative anionic surfactants include salts (for example alkaline salts, such as sodium salts, ammonium salts, amine salts, amino alcohol salts and magnesium salts) of the following compounds: alkyl sulfates, alkyl ether sulfates, alkylamidoether sulfates, alkylarylpolyether sulfates, monoglyceride sulfates; alkyl sulfonates, alkyl phosphates, alkylamide sulfonates, alkylaryl sulfonates, a-olefin sulfonates, paraffin sulfonates; alkyl sulfosuccinates, alkyl ether sulfosuccinates, alkylamide sulfosuccinates; alkyl sulfosuccinamates; alkyl sulfoacetates; alkyl ether phosphates; acyl sarcosinates; acyl isethionates and N-acyltaurates. The alkyl and acyl radicals of all of these various compounds can, for example, comprise from 12 to 20 carbon atoms, and the aryl radicals can, for example, be chosen from phenyl and benzyl groups.

For example, anionic surfactants can be chosen from fatty acid salts such as the salts of oleic, ricinoleic, palmitic and stearic acids, coconut oil acid and hydrogenated coconut oil acid and acyl lactylates in which the acyl radical comprises from 8 to 20 carbon atoms. At least one weakly anionic surfactant can also be used, such as alkyl-D-galactosiduronic acids and their salts, as well as polyoxyalkylenated (C₆-C₂₄) alkyl ether carboxylic acids, polyoxyalkylenated (C₆-C₂₄) alkylaryl ether carboxylic acids, polyoxyalkylenated (C₆-C₂₄) alkylamido ether carboxylic acids and their salts, for example, those comprising from 2 to 50 ethylene oxide groups.

As a further example, the anionic surfactant can be at least one salt chosen from alkyl sulfate salts and alkyl ether sulfate salts.

(ii) Nonionic surfactant(s):

Useful nonionic surfactants include compounds that are well known per se (see for example in this respect “Handbook of Surfactants” by M. R. Porter, published by Blackie & Son (Glasgow and London), 1991, pp. 116-178) the disclosure of which is incorporated by reference herein. Suitable nonionic surfactants can include, for example, polyethoxylated, polypropoxylated and polyglycerolated fatty acids, alkylphenols, a-diols and alcohols comprising a fatty chain comprising, for example, 8 to 18 carbon atoms, it being possible for the number of ethylene oxide and propylene oxide groups to range, for example, from 2 to 50 and for the number of glycerol groups to range, for example, from 2 to 30. Mention may also be made of copolymers of ethylene oxide and of propylene oxide, condensates of ethylene oxide and of propylene oxide with fatty alcohols; polyethoxylated fatty amides, for example, comprising from 2 to 30 mol of ethylene oxide, polyglycerolated fatty amides comprising on average 1 to 5, such as from 1.5 to 4, glycerol groups; oxyethylenated fatty acid esters of sorbitan comprising from 2 to 30 mol of ethylene oxide; fatty acid esters of sucrose, fatty acid esters of polyethylene glycol, alkylpolyglycosides, N-alkylglucamine derivatives, amine oxides such as (C₁₀-C₁₄)alkylamine oxides and N-acylaminopropylmorpholine oxides.

(iii) Amphoteric surfactant(s):

Representative amphoteric surfactants include surfactants chosen from aliphatic secondary and alphatic tertiary amine derivatives in which the aliphatic radical is chosen from linear and branched chain radicals comprising 8 to 22 carbon atoms and comprising at least one water-soluble anionic group (chosen, for example, from carboxylate, sulfonate, sulfate, phosphate and phosphonate); mention may also be made of (C₈-C₂₀)alkylbetaines, sulfobetaines, (C₈-C₂₀)alkylamido(C₁-C₆)alkylbetaines and (C₈-C₂₀)alkylamido(C₁-C₆)alkylsulfobetaines.

Representative amine derivatives include the products sold under the name Miranol, as described in U.S. Pat. Nos. 2,528,378 and 2,781,354, the disclosures of which are incorporated by reference herein, and having the structures:

R₂—CONHCH₂CH₂—N(R₃)(R₄)(CH₂COO—) (2)

in which:

R₂ is chosen from alkyl radicals derived from an acid R₂—COOH present in hydrolysed coconut oil, heptyl, nonyl and undecyl radicals,

R₃ is chosen from β-hydroxyethyl groups, and

R₄ is chosen from carboxymethyl groups;

and

R₅—CONHCH₂CH₂—N(B)(C) (3)

in which:

(B) is —CH₂CH₂OX′, with X′ chosen from a —CH₂CH₂—COOH group and a hydrogen atom,

(C) is —(CH₂)_{z}—Y′, with z=1 or 2, and with Y′ chosen from —COOH and —CH₂—CHOH—SO₃H radicals,

R₅ is chosen from alkyl radicals such as (a) alkyl radicals of an acid R₉—COOH present in oils chosen from coconut oil and hydrolysed linseed oil, (b) alkyl radicals, such as C₇, C₉, C₁₁ and C₁₃ alkyl radicals, and (c) C₁₇ alkyl radicals and the iso forms, and unsaturated C₁₇ radicals.

Such representative compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid, and cocoamphodipropionic acid.

By way of example, mention may be made of the cocoamphodiacetate sold under the trade name Miranol C2M Concentrate by the company Rhodia.

In the compositions in accordance with the present invention, at least two surfactants of different types may be used. Representative compositions include compositions comprising (a) more than one anionic surfactant, (b) at least one anionic surfactant and at least one amphoteric surfactant, and (c) at least one anionic surfactant and at least one nonionic surfactant. In one embodiment, the composition can comprise at least one anionic surfactant and at least one amphoteric surfactant.

The at least one anionic surfactant can, for example, be chosen from (C₁₂-C₁₄)alkyl sulfates of sodium, of triethanolamine and of ammonium; the (C₁₂-C₁₄)alkyl ether sulfates of sodium oxyethylenated with 2.2 mol of ethylene oxide; sodium cocoyl isethionate; and sodium (C₁₄-C₁₆)-a-olefin sulfonate, and used in combination with at least one amphoteric surfactant chosen from:

amphoteric surfactants such as the amine derivatives known as disodium cocoamphodipropionate and sodium cocoamphopropionate, sold, for example, by the company Rhodia under the trade name “Miranol C2M Conc.” as an aqueous solution comprising 38% active material, and under the name Miranol C32; and

amphoteric surfactants of zwitterionic type, such as alkylbetaines, for example the cocoylbetaine sold under the name “Dehyton AB 30” as an aqueous solution comprising 32% AM by the company Henkel and alkylamidobetaines such as Tegobetaine F50 sold by the company Goldschmidt.

The composition according to the present invention comprises at least one amphoteric starch chosen from the compounds of the following formulae:

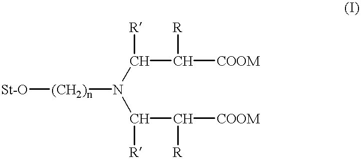

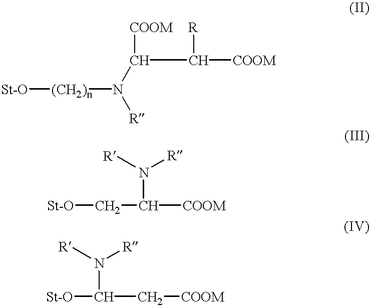

wherein:

St—O is a starch moiety;

R, which may be identical or different, are each chosen from a hydrogen atom and a methyl group;

R′, which may be identical or different, are each chosen from a hydrogen atom, a methyl group, and a —COOH group;

n is chosen from integers ranging from 2 to 3;

M, which may be identical or different, are each chosen from a hydrogen atom, an alkali metal, an alkaline-earth metal (such as Na, K, and Li), NH₄, quaternary ammonium compounds, and organic amines; and

R″, which may be identical or different, are each chosen from a hydrogen atom and alkyl groups comprising from 1 to 18 carbon atoms.

These compounds are disclosed, for example, in U.S. Pat. Nos. 5,455,340 and 4,017,460, the disclosures of which are incorporated herein by reference.

The starch moieties may, for example, be derived from any plant sources of starch such as, for example, corn, potato, oat, rice, tapioca, sorghum, barley and wheat. The starch hydrolysates mentioned above may also be used. For example, in one embodiment the starch is derived from potato.

Certain embodiments of the present invention comprise the starches of formulae (I) and (II). Additional embodiments comprise starches modified with 2-chloroethyl-aminodipropionic acid, i.e., the starches of formulae (I) and (II) in which R, R′ and R″ represent a hydrogen atom and n is equal to 2.

The at least one amphoteric starch according to the present invention can be used in the compositions in accordance with the present invention in amounts ranging for example from 0.01% to 10% by weight, such as from 0.1% to 5% by weight, relative to the total weight of the composition.

In one embodiment, the composition further comprises at least one cationic polymer.

As used herein, “cationic polymer” refers to polymers chosen from polymers comprising at least one cationic group and polymers comprising at least one group which can be ionized to form cationic groups.

The cationic polymers which may be used in accordance with the present invention may be chosen from any of those already known to improve at least one cosmetic property of hair treated with detergent compositions, such as, for example, those described in patent application EP-A-0 337,354 and in French patent applications FR-A-2 270,846, 2,383,660, 2,598,611, 2,470,596 and 2,519,863, the disclosures of which are incorporated herein by reference.

According to the present invention, the at least one cationic polymer may be chosen from polymers which comprise at least one unit comprising at least one group chosen from primary amine groups, secondary amine groups, tertiary amine groups and quaternary amine groups, wherein said at least one group forms part of the polymer skeleton, or is carried by at least one lateral substituent on said polymer skeleton.

According to the present invention, the at least one cationic polymer has a number-average molecular mass ranging for example from 500 to 5×10⁶, such as from 1×10³ to 3×10^{6.}

The at least one cationic polymer may be chosen from polymers of quaternary polyammonium, polymers of polyamino amide, and polymers of polyamine. Such polymers are known in the art.

For example, polymers of quaternary polyammonium, polymers of polyamino amide, and polymers of polyamine, which can be used in accordance with the present invention comprise the polymers described in French patents Nos. 2,505,348 and 2,542,997, the disclosures of which are incorporated herein. Non-limiting examples of such polymers include:

(1) Homo- and co-polymers derived from at least one monomer chosen from acrylic esters, methacrylic esters and amides, wherein said homo- and co-polymers comprise at least one unit chosen from units of formulae:

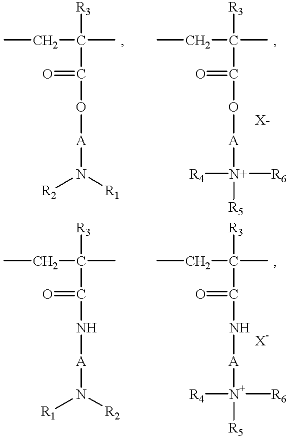

wherein:

R₃, which may be identical or different, are each chosen from a hydrogen atom and a CH₃ group;

A, which may be identical or different, are each chosen from linear and branched alkyl groups comprising from 1 to 6 carbon atoms, such as 2 or 3 carbon atoms, and hydroxyalkyl groups comprising from 1 to 4 carbon atoms;

R₄, R₅ and R₆, which may be identical or different, are each chosen from alkyl groups comprising from 1 to 18 carbon atoms, such as from 1 to 6 carbon atoms, and benzyl groups;

R₁ and R₂, which may be identical or different, are each chosen from a hydrogen atom and alkyl groups comprising from 1 to 6 carbon atoms, such as methyl and ethyl; and

X⁻ is an anion chosen from anions derived from at least one inorganic acid and anions derived from at least one organic acid, such as methosulfate anions, and halide atoms, such as chloride atoms and bromide atoms.

Copolymers of family (1) may further comprise at least one unit derived from at least one comonomer chosen from acrylamides, methacrylamides, diacetone acrylamides, acrylamides and methacrylamides, wherein said at least one comonomer is substituted on the nitrogen with at least one group chosen from lower (C₁-C₄) alkyls, acrylic acids, methacrylic acids, acrylic esters, methacrylic esters, vinyllactams and vinyl esters. Non-limiting examples of vinyllactams include vinylpyrrolidone and vinylcaprolactam.

Non-limiting examples of suitable copolymers include:

copolymers derived from at least one monomer of (i) acrylamide and (ii) dimethylaminoethyl methacrylate quaternized with at least one group chosen from dimethyl sulfate and dimethyl halide, such as the product sold under the name Hercofloc by the company Hercules;

copolymers derived from at least one monomer of (i) acrylamide and (ii) methacryloyloxyethyltrimethylammonium chloride described, for example, in patent application EP-A-080,976, the disclosure of which is incorporated herein by reference, and which is sold under the name Bina Quat P 100 by the company Ciba Geigy;

copolymers derived from at least one monomer of (i) acrylamide and (ii) methacryloyloxyethyltrimethylammonium methosulfate, such as, for example, copolymers sold under the name Reten by the company Hercules;

quaternized and non-quaternized vinylpyrrolidone/dialkylaminoalkyl acrylate copolymers and quaternized and non-quaternized vinylpyrrolidone/dialkylaminoalkyl methacrylate copolymers, such as the products sold under the name “Gafquat” by the company ISP, such as, for example, “Gafquat 734” or “Gafquat 755” and the products known as “Copolymer 845, 958 and 937”. These polymers are described in detail in French patents 2,077,143 and 2,393,573, the disclosures of which are incorporated herein by reference;

dimethylaminoethyl methacrylate/vinylcaprolactam/vinylpyrrolidone terpolymers, such as the product sold under the name Gaffix VC 713 by the company ISP;

vinylpyrrolidone/methacrylamidopropyidimethylamine copolymers, such as the product sold under the name Styleze CC 10 by ISP; and

quaternized vinylpyrrolidone/dimethylaminopropylmethacrylamide copolymers, such as the product sold under the name “Gafquat HS 100” by the company ISP.

(2) Cellulose ether derivatives comprising quaternary ammonium groups, such as those described in French patent 1,492,597, the disclosure of which is incorporated herein by reference, and polymers sold under the names “JR” (JR 400, JR 125 and JR 30M) and “LR” (LR 400, or LR 30M) by the company Union Carbide Corporation. These polymers are also defined in the CTFA dictionary as quaternary ammoniums of hydroxyethylcellulose which has reacted with an epoxide substituted with a trimethylammonium group.

(3) Cationic cellulose derivatives such as cellulose copolymers and cellulose derivatives grafted with at least one water-soluble monomer of quaternary ammonium, such as those described in U.S. Pat. No. 4,131,576, the disclosure of which is incorporated herein by reference, such as hydroxyalkylcelluloses (such as, for example, hydroxymethylcelluloses, hydroxyethylcelluloses and hydroxypropylcelluloses, wherein said hydroxyalkylcelluloses are grafted with at least one salt chosen from, for example, methacryloylethyltrimethylammonium salts, methacrylamidopropyltrimethylammonium salts and dimethyidiallylammonium salts). For example, commercial products corresponding to the aforementioned cationic cellulose derivatives include the products sold under the names “Celquat L 200” and “Celquat H 100” by the company National Starch.

(4) Cationic polysaccharides, such as those described in U.S. Pat. Nos. 3,589,578 and 4,031,307, the disclosures of which are incorporated herein by reference, such as guar gums comprising at least one cationic trialkylammonium group. For example, guar gums modified with at least one salt, such as a chloride salt, of 2,3-epoxypropyltrimethylammonium may be used in the present invention. Such products are sold in particular under the trade names Jaguar C13 S, Jaguar C 15, Jaguar C 17 and Jaguar C 162 by the company Meyhall.

(5) Polymers comprising (i) at least one piperazinyl unit and (ii) at least one group chosen from divalent alkylene groups and divalent hydroxyalkylene groups, wherein said at least one group optionally comprises at least one chain chosen from straight chains and branched chains, wherein said at least one chain is optionally interrupted by at least one entity chosen from oxygen atoms, sulfur atoms, nitrogen atoms, aromatic rings and heterocyclic rings, the oxidation products of said polymers and the quaternization products of said polymers. For example, such polymers are described in French patents 2,162,025 and 2,280,361, the disclosures of which are incorporated herein by reference.

(6) Water-soluble polyamino amides which may be prepared via at least one polycondensation reaction of at least one acidic compound and at least one polyamine compound, wherein said polyamino amides may be crosslinked with at least one crosslinking agent chosen from epihalohydrins, diepoxides, dianhydrides, unsaturated dianhydrides, bis-unsaturated derivatives, bis-halohydrins, bis-azetidiniums, bis-haloacyldiamines, bis-alkyl halides and oligomers derived from reaction of at least one difunctional compound with at least one compound chosen from bis-halohydrins, bis-azetidiniums, bis-haloacyidiamines, bis-alkyl halides, epihalohydrins, diepoxides and bis-unsaturated derivatives, wherein said crosslinking agent may be used in a proportion ranging for example from 0.025 mol to 0.35 mol per amine group of said polyamino amide, wherein said polyamino amides may optionally be alkylated, and wherein if said polyamino amides comprise at least one tertiary amine group, said polyamino amides may optionally be quaternized. For example, such polymers are described in French patents 2,252,840 and 2,368,508, the disclosures of which are incorporated herein by reference.

(7) Polyamino amide derivatives derived from condensation of at least one polyalkylene polyamine with at least one polycarboxylic acid, followed by alkylation with at least one difunctional agent.

Non-limiting examples of such polyamino amide derivatives include adipic acid/dialkylaminohydroxyalkyldialkylenetriamine polymers wherein the alkyl group comprises from 1 to 4 carbon atoms, such as methyl groups, ethyl groups and propyl groups. For example, such polymers are described in French patent 1,583,363, the disclosure of which is incorporated herein by reference.

Other non-limiting examples of such derivatives include the adipic acid/dimethylaminohydroxypropyl/diethylenetriamine polymers sold under the name “Cartaretine F, F4 or F8” by the company Sandoz.

(8) Polymers derived from reaction of (i) at least one polyalkylene polyamine comprising two primary amine groups and at least one secondary amine group with (ii) at least one dicarboxylic acid chosen from diglycolic acid and saturated aliphatic dicarboxylic acids comprising from 3 to 8 carbon atoms. According to the present invention, the molar ratio of the at least one polyalkylene polyamine to the at least one dicarboxylic acid ranges for example from 0.8:1 to 1.4:1. The polyamino amide resulting from the above reaction may be reacted with epichlorohydrin in a molar ratio of epichlorohydrin to the at least one secondary amine group of the polyamino amide ranges for example from 0.5:1 to 1.8:1. For example, such polymers are described in U.S. Pat. Nos. 3,227,615 and 2,961,347, the disclosures of which are incorporated herein by reference.

Polymers of this type are sold in particular under the name “Hercosett 57” by the company Hercules Inc. and under the name “PD 170” or “Delsette 101” by the company Hercules in the case of adipic acid/epoxypropyl/diethylenetriamine copolymers.

(9) Cyclopolymers of alkyldiallylamine and cyclopolymers of dialkyldiallylammonium, such as homopolymers and copolymers comprising, as the main constituent of the chain, at least one unit chosen from units of formulae (Va) and (Vb):

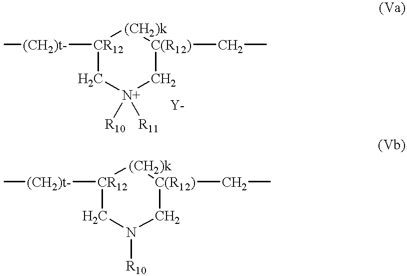

wherein:

k and t, which may be identical or different, are each chosen from 0 and 1, with the proviso that the sum of k+t is equal to 1;

R₁₂, which may be identical or different, are each chosen from a hydrogen atom and a methyl group;

R₁₀ and R₁₁, which may be identical or different, are each chosen from alkyl groups comprising from 1 to 22 carbon atoms, hydroxyalkyl groups wherein the alkyl portion of said hydroxyalkyl group optionally comprises from 1 to 5 carbon atoms, lower C₁-C₄ amidoalkyl groups, and, in addition,

R₁₀ and R₁₁, together with the nitrogen cation to which they are commonly attached, form at least one cationic heterocyclic group, such as cationic piperidyl groups and cationic morpholinyl groups;

Y⁻ is an anion, such as bromide, chloride, acetate, borate, citrate, tartrate, bisulfate, bisulfite, sulfate and phosphate. For example, such polymers are described in French patent 2,080,759 and in its Certificate of Addition 2,190,406, the disclosures of which are incorporated herein by reference.

In one embodiment, R₁₀ and R₁₁, which may be identical or different, are each chosen from alkyl groups comprising from 1 to 4 carbon atoms.

Non-limiting examples of the polymers defined above include the dimethyidiallyl-ammonium chloride homopolymer sold under the name “Merquat 100” by the company Calgon (and its homologues of low weight-average molecular mass) and copolymers of diallyldimethylammonium chloride and of acrylamide, sold under the name “Merquat 550”.

(10) Quaternary diammonium polymers comprising at least two repeating units of formula:

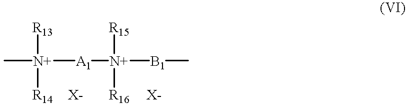

wherein:

R₁₃, R₁₄, R₁₅ and R₁₆, which may be identical or different, are each chosen from aliphatic groups comprising from 1 to 20 carbon atoms, alicyclic groups comprising from 1 to 20 carbon atoms, arylaliphatic groups comprising from 1 to 20 carbon atoms, lower hydroxyalkylaliphatic groups, and, in addition

at least two of said R₁₃, R₁₄, R₁₅ and R₁₆, together with the nitrogen cations to which they are attached, form at least one cationic heterocycle optionally comprising an additional heteroatom other than nitrogen, and, in addition,

R₁₃, R₁₄, R₁₅ and R₁₆, which may be identical or different, are each chosen from linear and branched C₁-C₆ alkyl groups substituted with at least one group chosen from nitrile groups, ester groups, acyl groups, amide groups and groups chosen from groups of formulae —CO—O—R₁₇—D and —CO—NH—R₁₇—D, wherein R₁₇ is chosen from alkylene groups and D is chosen from quaternary ammonium groups;

A₁ and B₁, which may be identical or different, are each chosen from polymethylene groups comprising from 2 to 20 carbon atoms chosen from linear and branched, saturated and unsaturated polymethylene groups wherein said polymethylene groups may optionally comprise, optionally linked to and optionally intercalated in the main chain, at least one entity chosen from aromatic rings, oxygen atoms, sulfur atoms, sulfoxide groups, sulfone groups, disulfide groups, amino groups, alkylamino groups, hydroxyl groups, quaternary ammonium groups, ureido groups, amide groups and ester groups; and

X⁻ is an anion chosen from anions derived from inorganic acids and anions derived from organic acids; and

A₁, R₁₃ and R₁₅ may optionally form, together with the two nitrogen atoms to which they are attached, at least one piperazine ring;

with the proviso that if A, is chosen from linear and branched, saturated and unsaturated alkylene groups and linear and branched, saturated and unsaturated hydroxyalkylene groups, B₁ may also be chosen from groups of formula:

(CH₂)ₙ—CO—D—OC—(CH₂)ₙ—

wherein D is chosen from:

a) glycol residues of formula: —O—Z—O—, wherein Z is chosen from linear and branched hydrocarbon groups and groups chosen from groups of formulae:

—(CH₂—CH₂—O)ₓ—CH₂—CH₂—; and

—[CH₂—CH(CH₃)-O]_{y}—CH₂—CH(CH₃)—

wherein x and y, which may be identical or different, are each chosen from integers ranging from 1 to 4 (in which case x and y represent a defined and unique degree of polymerization) and any number ranging from 1 to 4 (in which case x and y represent an average degree of polymerization);

b) bis-secondary diamine residues such as piperazine derivatives;

c) bis-primary diamine residues chosen from residues of formula: —NH—Y—NH—, wherein Y is chosen from linear and branched hydrocarbon groups and residues of formula

—CH₂—CH₂-S—S—CH₂—CH₂—; and

d) ureylene groups of formula: —NH—CO—NH—

wherein n in the above formula ranges from 1 to 6.

In one embodiment, X⁻ is an anion chosen from chloride ions and bromide ions.

According to the present invention, the quarternary diammonium polymers have a number-average molecular mass ranging for example from 1000 to 100,000.

For example, polymers of this type are described in French Patent Nos. 2,320,330, 2,270,846, 2,316,271, 2,336,434 and 2,413,907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020, the disclosures of which are incorporated herein by reference.

Further, according to the present invention, polymers comprising at least two repeating units of formula (VII) may be used:

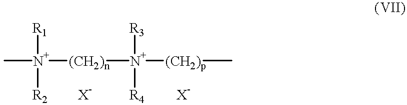

wherein:

R₁, R₂, R₃ and R₄, which may be identical or different, are each chosen from alkyl groups comprising from 1 to 4 carbon atoms and hydroxyalkyl groups comprising from 1 to 4 carbon atoms;

n and p, which may be identical or different, are each chosen from integers ranging from 2 to 20; and

X⁻ is an anion chosen from anions derived from inorganic acids and anoins derived from organic acids.

In one embodiment, R₁, R₂, R₃ and R₄ are chosen from methyl groups, n=3, p=6 and X⁻=Cl⁻. This unit is commonly known as Hexadimethrine chloride according to INCI 5 (CTFA) nomenclature.

(11) Polyquaternary ammonium polymers comprising at least one unit of formula (Vi11):

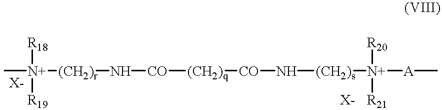

wherein:

R₁₈, R₁₉, R₂₀ and R₂₁which may be identical or different, are each chosen from a hydrogen atom, a methyl group, an ethyl group, a propyl group, a β-hydroxyethyl group, a β-hydroxypropyl group and —CH₂CH₂(OCH₂CH₂)ₚOH groups wherein p is an integer ranging from 0 to 6, with the proviso that R₁₈, R₁₉, R₂₀ and R₂₁ are all not simultaneously chosen from a hydrogen atom;

r and s, which may be identical or different, are each chosen from integers ranging from 1 to 6;

q is an integer ranging from 1 to 34;

X⁻ is an anion, such as a halide; and

A is chosen from dihalide groups and groups of formula —CH₂—CH₂—O—CH₂—CH₂—.

For example, such compounds are described in patent application EP-A-122,324, the disclosure of which is incorporated by reference.

Non-limiting examples of the polyquarternary ammonium polymers are “Mirapol A 15”, “Mirapol AD1”, “Mirapol AZ1” and “Mirapol 175” sold by the company Miranol.

(12) Quaternary polymers of vinylpyrrolidone and quaternary polymers of vinylimidazole, such as, for example, the products sold under the names Luviquat FC 905, FC 550 and FC 370 by the company BASF.

(13) Polyamines, such as Polyquart H sold by Henkel under the reference name “Polyethylene glycol (15) Tallow polyamine” in the CTFA dictionary.

(14) Crosslinked (meth)acryloyloxy(C₁-C₄)alkyltri(C₁-C₄)alkylammonium salt polymers, such as the polymers derived from homopolymerization of dimethylaminoethyl methacrylate quaternized with methyl chloride and polymers derived from copolymerization, for example, of acrylamide with dimethylaminoethyl methacrylate quaternized with a methyl chloride, wherein the homo- or copolymerization is followed by crosslinking with at least one compound comprising olefinic unsaturation, such as methylenebisacrylamide. For example, a crosslinked acrylamide/methacryloyloxyethyltrimethylammonium chloride copolymer (20/80 by weight) in the form of a dispersion comprising 50% by weight of the said copolymer in mineral oil may be used. This dispersion is sold under the name “Salcare SC 92” by the company Allied Colloids. Further, a crosslinked methacryloyloxyethyltrimethylammonium chloride homopolymer comprising 50% by weight of the homopolymer in mineral oil or in a liquid ester may be used. These dispersions are sold under the names “Salcare SC 95” and “Salcare SC 96” by the company Allied Colloids.

Other cationic polymers which may be used as the at least one cationic polymer according to the present invention are cationic proteins, cationic protein hydrolysates, polyalkyleneimines (such as polyethyleneimines), polymers comprising at least one vinylpyridine unit, polymers comprising at least one vinylpyridinium unit, condensates of polyamines, condensates of epichlorohydrin, quaternary polyureylenes and chitin derivatives.

In certain embodiments of the present invention, the at least one cationic polymer is chosen from quaternary cellulose ether derivatives (such as the products sold under the name “JR 400” by the company Union Carbide Corporation), cationic cyclopolymers (such as the homopolymers and copolymers of dimethyidiallylammonium chloride, sold under the names “Merquat 100”, “Merquat 550” and “Merquat S” by the company Calgon), cationic polysaccharides such as guar gums modified with a 2,3-epoxypropyltrimethylammonium salt, quaternary polymers of vinylpyrrolidone, and quaternary polymers of vinylimidazole.

The at least one cationic polymer is present in an amount ranging for example from 0.001% to 10% by weight, such as from 0.005% to 5% by weight, and further such as from 0.1% to 3% by weight, relative to the total weight of the final composition.

The compositions according to the present invention may further comprise at least one silicone.

The at least one silicone may be chosen from silicones that are soluble and insoluble in the composition. For example, the at least one silicone may be chosen from polyorganosiloxanes that are insoluble in the composition. The at least one silicone may also be in the form of at least one composition chosen from oils, waxes, resins and gums.

The organopolysiloxanes are defined in greater detail in the book by Walter Noll “Chemistry and Technology of Silicones” (1968) Academic Press, the disclosure of which is incorporated herein by reference. They can be chosen from volatile and non-volatile organopolysiloxanes.

When volatile, the at least one silicone can, for example, be chosen from silicones having a boiling point ranging for example from 60° C. to 260° C. Non-limiting examples of volatile silicones include:

(i) cyclic silicones comprising from 3 to 7 silicon atoms, such as from 4 to 5 silicon atoms. Non-limiting examples include octamethylcyclotetrasiloxane sold under the name “Volatile Silicone 7207” by Union Carbide, and “Silbione 70045 V 2” by Rhodia; and decamethylcyclopentasiloxane sold under the name “Volatile Silicone 7158” by Union Carbide, and “Silbione 70045 V 5” by Rhodia.

Mention may also be made of cyclocopolymers of the dimethylsiloxane/methylalkylsiloxane type, such as “Volatile Silicone FZ 3109” sold by the company Union Carbide, of chemical structure:

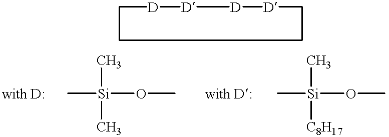

Mention may also be made of compositions comprising at least one cyclic silicone and at least one organosilicon compound, such as the composition comprising octamethylcyclotetrasiloxane and tetratrimethylsilylpentaerythritol (50/50) and the composition comprising octamethylcyclotetrasiloxane and oxy-1,l′-bis(2,2,2′,2′,3,3′-hexatrimethylsilyloxy)neopentane; and

(ii) linear volatile silicones comprising 2 to 9 silicon atoms with a kinematic viscosity of up to 5×10⁻⁶ m²/s at 25° C. Examples include decamethyltetrasiloxane sold under the name “SH 200” by the company Toray Silicone. Silicones forming part of this category are also described in the article published in Cosmetics and Toiletries, Vol. 91, Jan. 76, pp. 27-32, Todd & Byers “Volatile Silicone Fluids for Cosmetics,” the disclosure of which is incorporated herein by reference.

At least one non-volatile silicone can also be used. For example, the at least one non-volatile silicone can be chosen from polyalkylsiloxanes, polyarylsiloxanes, polyalkylarylsiloxanes, silicone gums, silicone resins, and polyorganosiloxanes modified with organofunctional groups.

The at least one non-volatile silicone may be chosen from polyalkylsiloxanes, among which mention may be made of polydimethylsiloxanes comprising trimethylsilyl end groups with a kinematic viscosity ranging for example from 5×10⁻⁶ to 2.5 m²/s at 25° C., such as a kinematic viscosity of 1×10⁻⁵ to 1 m²/s. The kinematic viscosity of the silicones is measured, for example, at 25° C. according to ASTM standard 445 Appendix C.

Non-limiting examples of polyalkylsiloxanes include the following commercial products:

the Silbione oils of the series 47 and 70,047 and the Mirasil oils sold by Rhodia, such as, for example, the oil 70,047 V 500,000;

the oils of the Mirasil series sold by the company Rhodia;

the oils of the 200 series from the company Dow Corning, such as, for example, DC200 with a kinematic viscosity of 60 000 Cst; and

the Viscasil oils from General Electric and certain oils of the SF series (SF 96, SF 18) from General Electric.

Mention may also be made of polydimethylsiloxanes comprising dimethylsilanol end groups (Dimethiconol according to the CTFA name) such as the oils of the 48 series from the company Rhodia.

Mention may additionally be made of the poly(C₁-C₂₀)alkylsiloxanes products sold under the names “Abil Wax 9800 and 9801” by the company Goldschmidt.

The at least one non-volatile silicone may be chosen from polyalkylarylsiloxanes chosen, for example, from linear and branched polydimethyl-methylphenylsiloxanes and polydimethyidiphenylsiloxanes, with a kinematic viscosity ranging for example from 1×10⁻⁵ to 5×10⁻² m²/s at 25° C.

Non-limiting examples of polyalkylarylsiloxanes include the products sold under the following names:

the Silbione oils of the 70,641 series from Rhodia;

the oils of the Rhodorsil 70,633 and 763 series from Rhodia;

the oil Dow Corning 556 Cosmetic Grade Fluid from Dow Corning;

the silicones of the PK series from Bayer, such as the product PK20;

the silicones of the PN and PH series from Bayer, such as the products PN 1000 and PH1000; and

certain oils of the SF series from General Electric, such as SF 1023, SF 1154, SF 1250 and SF 1265.

The at least one non-volatile silicone may be chosen from silicone gums, for example, polydiorganosiloxanes with high number-average molecular masses ranging for example from 200,000 to 1,000,000, used alone and in combination with at least one solvent. The at least one solvent can be chosen from volatile silicones, polydimethylsiloxane (PDMS) oils, polyphenylmethylsiloxane (PPMS) oils, isoparaffins, polyisobutylenes, methylene chloride, pentane, dodecane and tridecane.

Mention may also be made of the following products:

polydimethylsiloxane,

polydimethylsiloxane/methylvinylsiloxane gums,

polydimethylsiloxane/diphenylsiloxane,

polydimethylsiloxane/phenylmethylsiloxane, and

polyd imethylsiloxane/diphenylsiloxane/methylvinylsiloxane.

Other products which can be used in accordance with the present invention include:

compositions comprising at least one polydimethylsiloxane hydroxylated at the end of a chain (referred to as dimethiconol according to the nomenclature in the CTFA dictionary) and at least one cyclic polydimethylsiloxane (referred to as cyclomethicone according to the nomenclature in the CTFA dictionary), such as the product Q2 1401 sold by the company Dow Corning;

compositions comprising at least one polydimethylsiloxane gum and at least one cyclic silicone, such as the product SF 1214 Silicone Fluid from the company General Electric; this product is an SF 30 gum corresponding to a dimethicone, having a number-average molecular weight of about 500,000, dissolved in SF 1202 Silicone Fluid oil corresponding to decamethylcyclopentasiloxane;

compositions comprising combinations of PDMSs of different viscosities, such as combinations of at least one PDMS gum and at least one PDMS oil, for example, the product SF 1236 from the company General Electric. The product SF 1236 comprises a gum SE 30 defined above, having a kinematic viscosity of about 20 m²/s, and an oil SF 96, having a kinematic viscosity of about 5×10⁻⁶ m²/s. This product may generally comprise, for example, 15% SE 30 gum and 85% SF 96 oil.

The at least one non-volatile silicone may be chosen from organopolysiloxane resins which are crosslinked siloxane systems comprising at least one unit chosen from: R₂SiO_{2/2}, R₃SiO_{1/2}, RSiO_{3/2} and SiO_{4/2}, wherein:

R, which may be identical or different, are each chosen from hydrocarbon-based groups comprising 1 to 16 carbon atoms and phenyl groups. For example, in one embodiment, R is a C₁-C₄ lower alkyl group, such as a methyl group. In another embodiment R is a phenyl group. In another embodiment, the at least one non-volatile silicone may be chosen from organopolysiloxane resins which are crosslinked siloxane systems comprising the following units: R₂SiO_{2/2}, R₃SiO_{1/2}, RSiO_{3/2} and SiO_{4/2}.

Mention may also be made of the product sold under the name “Dow Corning 593” and those sold under the names “Silicone Fluid SS 4230 and SS 4267” by the company General Electric, which are silicones of dimethyl/trimethyl siloxane structure.

Mention may further be made of the resins of the trimethylsiloxysilicate type sold, for example, under the names X22-4914, X21-5034 and X21-5037 by the company Shin-Etsu.

The at least one non-volatile silicone may be chosen from organomodified silicones as defined above and comprising, in their structure, at least one organofunctional group attached via a hydrocarbon-based radical.

Among the organomodified silicones, mention may be made of polyorganosiloxanes comprising at least one constituent chosen from:

polyethylenoxy and polypropylenoxy groups optionally comprising at least one C₆-C₂₄ alkyl group, such as the products known as dimethicone copolyol sold by the company Dow Corning under the name DC 1248 and the oils Silwet L 722, L 7500, L 77 and L 711 from the company Union Carbide and the (C₁₂)alkylmethicone copolyol sold by the company Dow Corning under the name Q2 5200;

substituted and unsubstituted amine groups, such as the products sold under the name GP 4 Silicone Fluid and GP 7100 by the company Genesee, and the products sold under the names Q2 8220 and Dow Corning 929 and 939 by the company Dow Corning. The substituted amine groups are, for example, C₁-C₄ aminoalkyl groups;

thiol groups, such as the products sold under the names “GP 72 A” and “GP 71” from Genesee;

alkoxylated groups, such as the product sold under the name “Silicone Copolymer F-755” by SWS Silicones and Abil Wax 2428, 2434 and 2440 by the company Goldschmidt;

hydroxyl groups such as the polyorganosiloxanes comprising a hydroxyalkyl function, described in French patent application FR-A-85/16334, the disclosure of which is incorporated herein by reference;

acyloxyalkyl groups such as, for example, the polyorganosiloxanes described in U.S. Pat. No. 4,957,732, the disclosure of which is incorporated herein by reference;

anionic groups of carboxylic type, such as, for example, the products from the company Chisso Corporation described in patent EP 186,507, the disclosure of which is incorporated herein by reference, and of alkylcarboxylic type, such as those present in the product X-22-3701 E from the company Shin-Etsu; 2-hydroxyalkyl sulfonate; and 2-hydroxyalkyl thiosulfate such as the products sold by the company Goldschmidt under the names “Abil S201” and “Abil S255”; and

hydroxyacylamino groups, such as the polyorganosiloxanes described in patent application EP 342,834, the disclosure of which is incorporated herein by reference. Mention may be made, for example, of the product Q2-8413 from the company Dow Corning.

According to the present invention, silicones may also be used comprising a polysiloxane portion and a portion comprising a non-silicone organic chain, one of the two portions comprising the main chain of the polymer, the other being grafted onto the said main chain. These polymers are described, for example, in patent applications EP-A-412,704, EP-A-412,707, EP-A-640,105, WO 95/00578, EP-A-582,152 and WO 93/23009 and U.S. Pat. Nos. 4,693,935, 4,728,571 and 4,972,037, the disclosures of which are incorporated herein by reference. These polymers may be chosen from anionic and nonionic polymers.

Such polymers are, for example, copolymers which can be obtained by radical polymerization of a monomer composition comprising:

a) from 50% to 90% by weight tert-butyl acrylate;

b) from 0% to 40% by weight acrylic acid; and

c) from 5% to 40% by weight silicone macromer of formula:

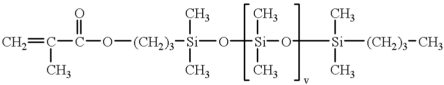

wherein v is a number ranging from 5 to 700; the weight percentages are relative to the total weight of the monomers.

Other examples of grafted silicone polymers include polydimethylsiloxanes (PDMS) onto which are grafted, via a connecting chain unit of thiopropylene type, at least one mixed polymer unit of poly(meth)acrylic acid type and of at least one mixed polymer unit of polyalkyl (meth)acrylate type; and polydimethylsiloxanes (PDMS) onto which are grafted, via a connecting chain unit of thiopropylene type, at least one polymer unit of polyisobutyl (meth)acrylate type.

According to the present invention, all of the silicones may also be used in the form of emulsions, nanoemulsions and microemulsions.

Other non-limiting examples of polyorganosiloxanes include:

non-volatile silicones chosen from the family of polyalkylsiloxanes comprising trimethylsilyl end groups, such as oils with a kinematic viscosity ranging for example from 0.2 to 2.5 m²/s at 25° C., such as the oils of the series DC200 from Dow Corning, and further such as, oils with a kinematic viscosity of 60,000 Cst, of the series Silbione 70047 and 47 and the oil 70,047 V 500,000, which are sold by the company Rhodia, polyalkylsiloxanes comprising dimethylsilanol end groups, such as dimethiconol, and polyalkylarylsiloxanes such as the oil Silbione 70641 V 200 sold by the company Rhodia;

the organopolysiloxane resin sold under the name Dow Corning 593;

polysiloxanes comprising at least one amine group, such as amodimethicones and trimethylsilylamodimethicone.

According to the present invention, the at least one silicone can be present in amounts ranging for example from 0.001% to 20% by weight, such as from 0.01% to 10% by weight, and further such as from 0.1% to 3% by weight, relative to the total weight of the final composition.

The cosmetically acceptable aqueous medium can comprise water. In another embodiment, the cosmetically acceptable aqueous medium can comprise water and at least one cosmetically acceptable solvent such as a C₁-C₄ alcohol chosen from, for example, ethanol, isopropanol, tert-butanol and n-butanol; alkylene glycols, for example propylene glycol, and glycol ethers.

The compositions according to the present invention have a final pH ranging for example from 3 to 10, such as from 4 to 8. The pH can be adjusted to the desired value by adding at least one base (chosen from organic and inorganic bases) to the composition, such as, for example, bases chosen from aqueous ammonia and primary, secondary and tertiary (poly)amines, for example monoethanolamine, diethanolamine, triethanolamine, isopropanolamine and 1,3-propanediamine. The pH can also be adjusted to the desired value by adding at least one acid, such as, for example, a carboxylic acid such as, for example, citric acid.

The compositions in accordance with the present invention may optionally comprise at least one viscosity modifier such as viscosity modifiers chosen from electrolytes and thickeners (which can be chosen from associative and non-associative thickeners). Mention may be made, for example, of sodium chloride, sodium xylenesulfonate, scleroglucans, xanthan gums, fatty acid alkanolamides, alkyl ether carboxylic acid alkanolamides optionally oxyethylenated with up to 5 mol of ethylene oxide, such as the product sold under the name “Aminol A 15” by the company Chem Y, crosslinked polyacrylic acids and crosslinked acrylic acid/C₁₀-C₃₀ alkyl acrylate copolymers. The at least one viscosity modifier may be present in the compositions according to the present invention in amounts ranging for example from 0% to 10% by weight, relative to the total weight of the composition.

The compositions in accordance with the present invention may optionally comprise at least one additive chosen from nacreous agents and opacifiers, wherein said additive may be present in amounts ranging for example from 0% to 5% by weight, relative to the total weight of the final composition. Non-limiting examples include, C₁₆ higher fatty alcohols, fatty-chain acyl derivatives such as the monostearates and distearates of ethylene glycol and the monostearates and distearates of polyethylene glycol, and fatty-chain ethers such as, for example, distearyl ether and 1-(hexadecyloxy)-2-octadecanol.

The compositions in accordance with the present invention may also optionally comprise at least one additive chosen from foam synergists such as C₁₀-C₁₈ 1,2-alkanediols and fatty alkanolamides derived from monoethanolamine, C₁₀-C₁₈ 1,2-alkanediols and fatty alkanolamides derived from from diethanolamine, silicone sunscreens, non-silicone sunscreens, cationic surfactants, anionic polymers, nonionic polymers, amphoteric polymers, proteins, protein hydrolysates, ceramides, pseudoceramides, fatty acids comprising at least one chain chosen from linear and branched C₁₂-C₄₀ chains such as 18-methyleicosanoic acid, hydroxy acids, vitamins, provitamins such as panthenol, plant, animal, mineral and synthetic oils and any other additive conventionally used in the cosmetics field which does not adversely affect the at least one advantageous property of the compositions according to the present invention.

Needless to say, a person skilled in the art will take care to select any optional additional compound(s) and the amounts thereof such that the at least one advantageous property intrinsically associated with the composition according to the present invention is not substantially, adversely affected by the envisaged addition(s).

These additives may be present in the composition according to the present invention in amounts ranging for example from 0% to 20% by weight relative to the total weight of the composition. The amount of each additive can be determined by a person skilled in the art according to its nature and its function.

The compositions according to the present invention, may be in the form of at least one composition chosen from, for example, thickened liquids, creams, and gels, and they are, for example, suitable for washing and caring for keratin materials, such as the hair and the skin and further such as the hair.

When the compositions in accordance with the present invention are used as shampoos, they can be simply applied to wet hair and the foam generated by massaging or rubbing with the hands can be removed by rinsing out with water, after optionally being left to stand on the hair for a period of time, it being possible for the operation to be repeated at least one time.

A subject of the present invention is also a process for washing and conditioning keratin materials such as, for example, the hair, which comprises applying to the wet materials an effective amount of a composition as defined above, and then in rinsing out with water, after optionally leaving to stand on the hair for a period of time.

The compositions according to the present invention may be used as shampoos for washing and conditioning the hair and, in this case, they may be applied to moistened hair in amounts that are effective to wash it, this application being followed by rinsing with water.

The compositions according to the present invention may also be used as shower gels for washing and conditioning the hair and the skin, in which case they may be applied to moistened skin and hair and are rinsed off after application.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

Non-limiting examples illustrating the present invention are given below:

### EXAMPLE 1

A shampoo composition in accordance with the present invention (composition A) was prepared:

| Composition | Invention A |
|---|---|
| Sodium lauryl ether sulfate (70/30 C12/C14) comprising 2.2 | 15 g AM |
| mol of ethylene oxide, as an aqueous solution comprising | |
| 28% AM | |
| Potato starch modified with 2-chloroethylaminodipropionic | 0.5 g |
| acid neutralized with sodium hydroxide (Structure Solanace | |
| from National Starch) | |
| Hydroxyethylcellulose crosslinked with epichlorohydrin and | 0.4 g |
| quaternized with trimethylamine (JR 400 by the company | |
| Union Carbide) | |
| Hydroxyethylcellulose | 0.4 9 |
| Citric acid qs | pH 7 |
| Demineralized water qs | 100 g |

Shampooing was carried out by applying about 12 g of composition A to hair which had been moistened beforehand. The shampoo was worked into a lather and was then rinsed out thoroughly with water.

Tests showed that the composition according to the present invention gave, on wet hair, considerable volumizing and a great deal of lightness.

### EXAMPLES 2 AND 3

EXAMPLES 2 AND 3

| | Example 2 | Example 3 |
|---|---|---|
| Sodium lauryl ether sulfate (70/30 C12/C14) | 16 g AM | 16 g AM |
| comprising 2.2 mol of ethylene oxide | | |
| Cocoylamidopropylbetaine as an aqueous | 2 g AM | 2 g AM |
| solution comprising 38% A.M. | | |
| Hydroxyethylcellulose crosslinked with | 0.4 g | — |
| epichlorohydrin, quaternized with trimethylamine | | |
| (polymer JR 400 from the company Union | | |
| Carbide) | | |
| Hydroxypropyl guar trimethylammonium | — | 0.1 g |
| chloride, sold under the name Jaguar C13S by the | | |
| company Rhodia | | |
| Potato starch modified with | | |
| 2-chloroethylaminodipropionic acid neutralized | 0.2 g | 0.3 g |
| with sodium hydroxide (Structure Solanace from | | |
| National Starch) | | |
| Polydimethylsiloxane of kinematic viscosity | | |
| 300,000 cSt | | |
| Polydimethylsiloxane of kinematic viscosity | — | 2.7 g |
| 500,000 cSt | 1.5 g | — |
| 1-(hexadecyloxy)-2-octadecanol/cetyl alcohol | | |
| mixture | 3 g | 3 g |
| Coconut monoisopropanolamide | 0.5 g | 0.5 g |
| Preserving agents, fragrance | qs | qs |
| pH agent qs | pH 7 | pH 7 |
| Demineralized water qs | 100 g | 100 g |

Shampooing was carried out by applying about 12 g of the composition to hair which had been moistened beforehand. The shampoo was worked into a lather and was then rinsed out thoroughly with water.

The hair treated with the composition of Example 2 or of Example 3 was soft, light and disentangled easily.

### EXAMPLE 4

A shampoo in accordance with the present invention, having the composition below, was prepared:

| | |
|---|---|
| Sodium lauryl ether sulfate (70/30 C12/C14) comprising 2.2 mol of | 15.5 g AM |
| ethylene oxide | |
| Cocoylbetaine as an aqueous solution comprising 32% A.M. | 3 g AM |
| Hydroxypropyl guar trimethylammonium chloride, sold under the | 0.1 g |
| name Jaguar C13S by the company Rhodia | |
| Potato starch modified with 2-chloroethylaminodipropionic acid | |
| neutralized with sodium hydroxide (Structure Solanace from National | 0.3 g |
| Starch) | |
| Polydimethylsiloxane of kinematic viscosity 60,000 cSt | 2.7 g |
| Amodimethicone as a cationic emulsion comprising 35% A.M. | 1.05 g AM |
| (DC939 from Dow Corning) | |
| 1-(Hexadecyloxy)-2-octadecanol/cetyl alcohol mixture | 2.5 g |
| Coconut monoisopropanolamide | |
| Preserving agents, fragrance | 0.5 g |
| Citric acid qs | qs |
| Demineralized water qs | pH 5.5 |
| | 100 g |

Shampooing was carried out by applying about 12 g of the composition to hair which had been moistened beforehand. The shampoo was worked into a lather and was then rinsed out thoroughly with water.

The hair treated with this composition was soft, light and disentangled easily.

What is claimed is:

1. A cosmetic composition comprising, in a cosmetically acceptable aqueous medium, a washing base and at least one amphoteric starch chosen from the compounds of formulae (I) to (IV): 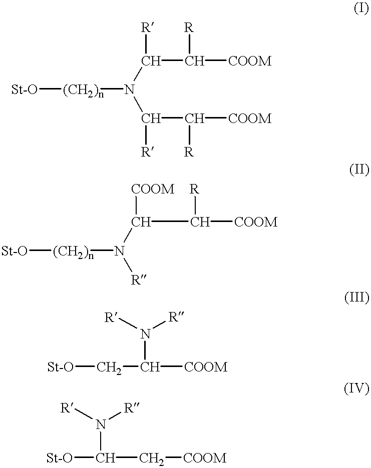
wherein:
St—O is a starch moiety;
R, which may be identical or different, are each chosen from a hydrogen atom and a methyl group;
R′, which may be identical or different, are each chosen from a hydrogen atom, a methyl group, and a —COOH group;
n is chosen from integers ranging from 2 to 3;
M, which may be identical or different, are each chosen from a hydrogen atom, an alkali metal, an alkaline-earth metal, NH₄, quaternary ammonium compounds, and organic amines; and
R″, which may be identical or different, are each chosen from a hydrogen atom and alkyl groups comprising from 1 to 18 carbon atoms, wherein said composition is a detergent and conditioning composition, and wherein said composition is free of fatty acid soaps.

2. A composition according to claim 1, wherein said at least one amphoteric starch is chosen from the compounds of formula (I) and (II).

3. A composition according to claim 2, wherein R, R′ and R″ are hydrogen and n is equal to 2.

4. A composition according to claim 1, wherein said washing base comprises at least one surfactant chosen from anionic, amphoteric and nonionic surfactants.

5. A composition according to claim 1, wherein said washing base comprises at least one anionic surfactant.

6. A composition according to claim 1, wherein said washing base is present in an amount ranging from 4% and 50% by weight, relative to the total weight of the composition.

7. A composition according to claim 6, wherein said washing base is present in an amount ranging from 6% to 35% by weight, relative to the total weight of the composition.

8. A composition according to claim 7, wherein said washing base is present in an amount ranging from 8% to 25% by weight, relative to the total weight of the composition.

9. A composition according to claim 1, wherein said at least one amphoteric starch is present in an amount ranging from 0.01% to 10% by weight, relative to the total weight of the composition.

10. A composition according to claim 9, wherein said at least one amphoteric starch is present in an amount ranging from 0.1% to 5% by weight, relative to the total weight of the composition.

11. A composition according to claim 1 further comprising at least one cationic polymer.

12. A composition according to claim 11, wherein said at least one cationic polymer is chosen from quaternary cellulose ether derivatives, cationic cyclopolymers, cationic polysaccharides, quaternary polymers of vinyl pyrrolidone and quaternary polymers of vinylimidazole.

13. A composition according to claim 12, wherein said cationic cyclopolymers are chosen from diallyldimethylammonium chloride homopolymers and copolymers of diallyldimethylammonium chloride and acrylamide.

14. A composition according to claim 12, wherein said quaternary cellulose ether derivatives are chosen from hydroxyethylcelluloses which have reacted with an epoxide substituted with a trimethylammonium group.

15. A composition according to claim 12, wherein said cationic polysaccharides are chosen from guar gums modified with a 2,3-epoxypropyltrimethylammonium salt.

16. A composition according to claim 11, wherein said at least one cationic polymer is present in an amount ranging from 0.001% to 10% by weight, relative to the total weight of the composition.

17. A composition according to claim 16, wherein said at least one cationic polymer is present in an amount ranging from 0.005% to 5% by weight, relative to the total weight of the composition.

18. A composition according to claim 17, wherein said at least one cationic polymer is present in an amount ranging from 0.01% to 3% by weight, relative to the total weight of the composition.

19. A composition according to claim 1 further comprising at least one silicone.

20. A composition according to claim 19, wherein said at least one silicone is chosen from non-volatile polyorganosiloxanes.

21. A composition according to claim 20, wherein said non-volatile polyorganosiloxanes are chosen from polyalkylsiloxanes, polyarylsiloxanes, polyalkylarylsiloxanes, silicone gums, silicone resins, and polyorganosiloxanes modified with organofunctional groups.

22. A composition according to claim 21, wherein said polyalkylsiloxanes are chosen from polydimethylsiloxanes comprising trimethylsilyl end groups, polydimethylsiloxanes comprising dimethylsilanol end groups, and poly(C₁-C₂₀)alkylsiloxanes.

23. A composition according to claim 21, wherein said polyalkylarylsiloxanes are chosen from
linear polydimethylmethylphenylsiloxanes,
branched polydimethylmethylphenylsiloxanes,
linear polydimethyldiphenylsiloxanes, and
branched polydimethylmethylphenylsiloxanes.

24. A composition according to claim 23, wherein said polyalkylarylsiloxanes have a kinematic viscosity ranging from 1×10⁻⁵ m²/s to 5×10⁻² m²/s at 25° C.

25. A composition according to claim 21, wherein said silicone gums are chosen from polydiorganosiloxanes with number-average molecular masses ranging from 200,000 to 1,000,000.

26. A composition according to claim 25, wherein said silicone gums are used alone or in combination with at least one solvent.

27. A composition according to claim 21, wherein said silicone resins are chosen from resins comprising at least one unit chosen from R₃SiO_{1/2}, R₂SiO_{2/2}, RSiO_{3/2}, and SiO_{4/2},
wherein R, which may be identical or different, are each chosen from hydrocarbon-based groups comprising 1 to 16 carbon atoms and phenyl groups.

28. A composition according to claim 27, wherein said silicone resins are chosen from resins comprising the following units: R₃SiO_{1/2}, R₂SiO_{2/2}, RSiO_{3/2}, and SiO_{4/2}.

29. A composition according to claim 21, wherein said organomodified silicones are chosen from silicones comprising, in their structure, at least one organofunctional group attached via a hydrocarbon-based radical.

30. A composition according to claim 19, wherein said at least one silicone is chosen from polyalkylsiloxanes comprising trimethylsilyl end groups, polyalkylsiloxanes comprising dimethylsilanol end groups, polyalkylarylsiloxanes, combinations of polydimethylsiloxaness comprising at least one gum and at least one oil of different viscosities, combinations of organosiloxanes and cyclic silicones, and polyorganosiloxane resins.

31. A composition according to claim 19, wherein said at least one silicone is present in an amount ranging from 0.001% to 20% by weight, relative to the total weight of the composition.

32. A composition according to claim 31, wherein said at least one silicone is present in an amount ranging from 0.01% and 10% by weight, relative to the total weight of the composition.

33. A composition according to claim 1 further comprising at least one additive chosen from C₁₀-C₁₈ 1,2-alkanediols and fatty alkanolamides derived from monoethanolamine, C₁₀-C₁₈ 1,2-alkanediols and fatty alkanolamides derived from from diethanolamine, silicone sunscreens, non-silicone sunscreens, cationic surfactants, anionic polymers, nonionic polymers, amphoteric polymers, proteins, protein hydrolysates, ceramides, pseudoceramides, fatty acids comprising at least one chain chosen from linear and branched C₁₂-C₄₀ chains, 18-methyleicosanoic acid, hydroxy acids, vitamins, provitamins, panthenol, plant oils, animal oils, mineral oils and synthetic oils.

34. A composition according to claim 33, wherein said at least one additive is present in an amount ranging from greater than 0% to 20% by weight, relative to the total weight of the composition.

35. A cosmetic composition comprising, in a cosmetically acceptable aqueous medium, a washing base and at least one amphoteric starch chosen from the compounds of formulae (I) to (IV): 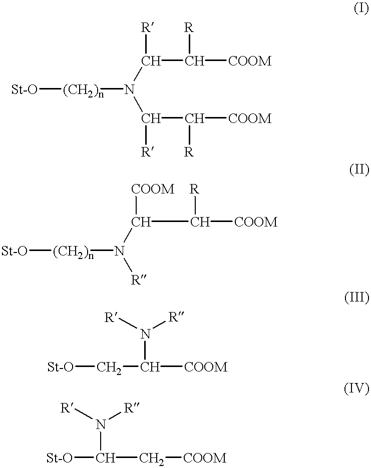
wherein:
St—O is a starch moiety;
R, which may be identical or different, are each chosen from a hydrogen atom and a methyl group;
R′, which may be identical or different, are each chosen from a hydrogen atom, a methyl group, and a —COOH group;
n is chosen from integers ranging from 2 to 3;
M, which may be identical or different, are each chosen from a hydrogen atom, an alkali metal, an alkaline-earth metal, NH₄, quaternary ammonium compounds, and organic amines; and
R″, which may be identical or different, are each chosen from a hydrogen atom and alkyl groups comprising from 1 to 18 carbon atoms, wherein said composition is a detergent composition, and wherein said composition is free of fatty acid soaps.

36. A cosmetic composition comprising, in a cosmetically acceptable aqueous medium, a washing base and at least one amphoteric starch chosen from the compounds of formulae (I) to (IV): 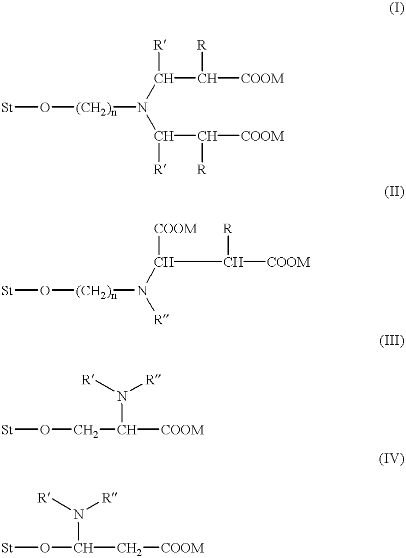
wherein:
St—O is a starch moiety;
R, which may be identical or different, are each chosen from a hydrogen atom and a methyl group;
R′, which may be identical or different, are each chosen from a hydrogen atom, a methyl group, and a —COOH group;
n is chosen from integers ranging from 2 to 3;
M, which may be identical or different, are each chosen from a hydrogen atom, an alkali metal, an alkaline-earth metal, NH₄, quaternary ammonium compounds, and organic amines; and
R″, which may be identical or different, are each chosen from a hydrogen atom and alkyl groups comprising from 1 to 18 carbon atoms, wherein said composition is a conditioning composition, and wherein said composition is free of fatty acid soaps.

37. A process for at least partially removing make-up from keratin materials comprising applying to said keratin materials an amount of a composition effective to at least partially remove said makeup, said composition comprising, in a cosmetically acceptable aqueous medium, a washing base and at least one amphoteric starch chosen from the compounds of formulae (I) to (IV): 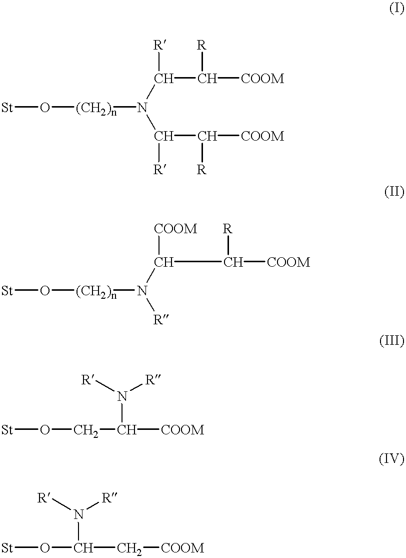
wherein:
St—O is a starch moiety;
R, which may be identical or different, are each chosen from a hydrogen atom and a methyl group;
R′, which may be identical or different, are each chosen from a hydrogen atom, a methyl group, and a —COOH group;
n is chosen from integers ranging from 2 to 3;
M, which may be identical or different, are each chosen from a hydrogen atom, an alkali metal, an alkaline-earth metal, NH₄, quaternary ammonium compounds, and organic amines; and
R″, which may be identical or different, are each chosen from a hydrogen atom and alkyl groups comprising from 1 to 18 carbon atoms, wherein said composition is free of fatty acid soaps.

38. A process according to claim 37, wherein said keratin materials are chosen from skin and hair.

39. A process for conditioning a keratin material comprising applying to said keratin material an amount of a composition effective to condition said keratin materials, said composition comprising, in a cosmetically acceptable aqueous medium, a washing base and at least one amphoteric starch chosen from the compounds of formulae (I) to (IV) 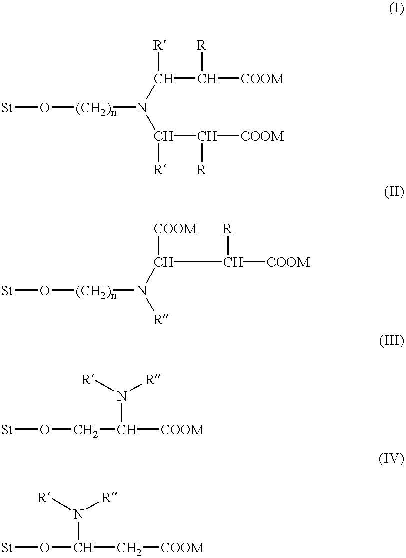
wherein:
St—O is a starch moiety;
R, which may be identical or different, are each chosen from a hydrogen atom and a methyl group;
R′, which may be identical or different, are each chosen from a hydrogen atom, a methyl group, and a —COOH group;
n is chosen from integers ranging from 2 to 3;
M, which may be identical or different, are each chosen from a hydrogen atom, an alkali metal, an alkaline-earth metal, NH₄, quaternary ammonium compounds, and organic amines; and
R″, which may be identical or different, are each chosen from a hydrogen atom and alkyl groups comprising from 1 to 18 carbon atoms, wherein said composition is free of fatty acid soaps.

40. A process according to claim 39, wherein said keratin material is hair.

41. A process for washing and conditioning a keratin material comprising:
(a) applying to said keratin material an effective amount of a composition to wash and condition said keratin material; and
(b) rinsing said keratin material with water, wherein said composition comprises, in a cosmetically acceptable aqueous medium, a washing base and at least one amphoteric starch chosen from the compounds of formulae (I) to (IV): 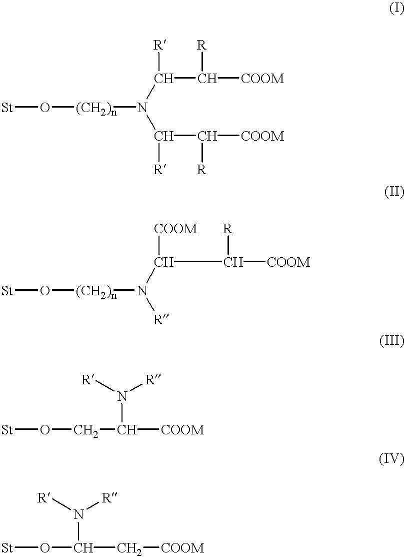
wherein:
St—O is a starch moiety;
R, which may be identical or different, are each chosen from a hydrogen atom and a methyl group;
R′, which may be identical or different, are each chosen from a hydrogen atom, a methyl group, and a —COOH group;
n is chosen from integers ranging from 2 to 3;
M, which may be identical or different, are each chosen from a hydrogen atom, an alkali metal, an alkaline-earth metal, NH₄, quaternary ammonium compounds, and organic amines; and
R″, which may be identical or different, are each chosen from a hydrogen atom and alkyl groups comprising from 1 to 18 carbon atoms, wherein said composition is free of fatty acid soaps.

42. A process according to claim 41, wherein said keratin material is wet before applying said composition.

43. A process according to claim 41, wherein said composition is left to stand on said keratin material for a period of time.

44. A process according to claim 41, wherein said keratin material is hair.
